(19) United States
(12) Patent Application Publication
(10) Pub. No.: US 2024/0009258 A1
(43) Pub. Date: Jan. 11, 2024

(54) ANTI-CANCER COMPOSITION AND USE THEREOF

(71) Applicants: Yao-Kuan CHEN, Taichung City (TW); DR. OXFORD BIOTECH FACTORY CO., LTD., Taichung City (TW)

(72) Inventors: Yao-Kuan CHEN, Taichung City (TW); Daniel Tzu-Hsuan CHEN, Taichung City (TW); Hui-Min CHIU, Taichung City (TW)

(21) Appl. No.: 18/162,038

(22) Filed: Jan. 31, 2023

(30) Foreign Application Priority Data
Jul. 8, 2022 (TW) 111125754

(51) Int. Cl. A61K 36/28; A61K 36/899; A61K 36/605; A61K 41/00; A61P 35/00

(52) U.S. Cl. CPC A61K 36/28; A61K 36/899; A61K 36/605; A61K 41/0057; A61P 35/00; A61K 2236/333

(57) ABSTRACT

Disclosed herein is an anti-cancer composition that includes an ethanol extract of green propolis, an ethanol extract of wheatgrass, and an ethanol extract of mulberry leaves. Also disclosed herein is use of the anti-cancer composition for inhibiting the growth of cancer cells.

### CROSS-REFERENCE TO RELATED APPLICATION

This application claims priority of Taiwanese Invention Patent Application No. 111125754, filed on Jul. 8, 2022.

### FIELD

The present disclosure relates to an anti-cancer composition including an ethanol extract of green propolis, an ethanol extract of wheatgrass, and an ethanol extract of mulberry leaves. The present disclosure also relates to a method for inhibiting the growth of cancer cells using the anti-cancer composition.

### BACKGROUND

Cancer is currently one of the leading causes of death worldwide. Carcinogenesis or tumorigenesis is mainly due to the accumulation of exogenous or endogenous factors in cells, resulting in genetic abnormalities that lead to errors in intracellular signaling pathways and cell cycle that is out of control, eventually forming cancer cells.

The current clinical treatment strategies for cancer include use of anti-cancer drugs and photodynamic therapy (PDT). PDT is performed by irradiating a photosensitizer (e.g., Photofrin) accumulated in cancer cells with a light source having a specific wavelength (e.g., 620 nm-750 nm), so as to excite the photosensitizer to produce singlet oxygen and free radicals that are toxic to cancer cells, thereby damaging the cancer cells. However, these treatment strategies might not be able to achieve the desired therapeutic effect and cure rate. The main reasons include individual differences in patients, severe side effects of anti-cancer drugs, and multi-drug resistance and metastatic ability of cancer cells.

Green propolis is a yellow-green gummy substance formed by mixing beeswax with the juice from plant buds, and is usually collected in summer. About 60,000 bees can only produce 100 g to 150 g of green propolis in a year, so the green propolis is very rare and precious, and is known as “green gold”. It has been reported that green propolis has anti-bacterial, anti-parasitic, and anti-inflammatory activities.

TW 1679015 B discloses a method for extracting green propolis, which includes subjecting green propolis to an extraction treatment with ethanol, followed by filtration, so as to obtain a filtrate of green propolis, subjecting the filtrate of green propolis to a concentration treatment to form a concentrate of green propolis, mixing the concentrate of green propolis with propylene glycol, followed by subjecting a mixture thus obtained to a dewaxing treatment with triglycerides, so as to obtain a green propolis extract. It can be seen from the examples of TW 1679015 B that the green propolis extract prepared by the method has high concentration and purity. However, TW 1679015 B does not disclose that the green propolis extract has an inhibitory effect on the growth of cancer cells.

In spite of the aforesaid, there is still a need to develop a new strategy that can be utilized for inhibiting the growth of cancer cells.

### SUMMARY

Therefore, in a first aspect, the present disclosure provides an anti-cancer composition which can alleviate at least one of the drawbacks of the prior art.

The anti-cancer composition includes an ethanol extract of green propolis, an ethanol extract of wheatgrass, and an ethanol extract of mulberry leaves.

In a second aspect, the present disclosure provides a method for inhibiting the growth of cancer cells, which can alleviate at least one of the drawbacks of the prior art, and which includes administering to a subject in need thereof the aforesaid anti-cancer composition.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the high performance liquid chromatography (HPLC) spectrum of the GP-WM extract;

FIG. 2 shows the cell viability percentages determined in the U87 cells treated with different amounts of a respective one of the GP extract, the WM extract, and the GP-WM extract;

FIG. 3 shows the cell viability percentages determined in the U87 cells treated with different amounts of a respective one of the GP extract, the WM extract, and the GP-WM extract in combination with photodynamic therapy (PDT); and

FIG. 4 shows the cell viability percentages determined in the 786-O cells treated with different amounts of the GP-WM extract.

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides an anti-cancer composition, which includes an ethanol extract of green propolis, an ethanol extract of wheatgrass, and an ethanol extract of mulberry leaves.

In certain embodiments, the ethanol extract of green propolis, the ethanol extract of wheatgrass, and the ethanol extract of mulberry leaves in the anti-cancer composition are present in a weight ratio ranging from 1:0.1:0.1 to 1:1:1. In an exemplary embodiment, the weight ratio of the ethanol extract of green propolis, the ethanol extract of wheatgrass, and the ethanol extract of mulberry leaves is 1:0.5:0.5.

According to the present disclosure, the ethanol extract of green propolis, the ethanol extract of wheatgrass, and the ethanol extract of mulberry leaves suitable for use in this disclosure are not particularly limited, and may be prepared using techniques well-known to those skilled in the art (for example, see TW 1679015 B).

It should be noted that the procedures and operating conditions for extracting the aforesaid ethanol extracts may be adjusted according to practical requirements, and are within the expertise and routine skills of those skilled in the to art.

According to the present disclosure, the ethanol extract of green propolis, the ethanol extract of wheatgrass, and the ethanol extract of mulberry leaves may be independently obtained by an extraction treatment that is conducted at a temperature ranging from 30° C. to 70° C. In certain embodiments, the extraction treatment may be conducted at a temperature ranging from 30° C. to 50° C.

In certain embodiments, the ethanol extract of green propolis, the ethanol extract of wheatgrass, and the ethanol extract of mulberry leaves may be independently dissolved in propylene glycol.

In certain embodiments, the ethanol extract of green propolis may be further subjected to a dewaxing treatment after being dissolved in propylene glycol.

According to the present disclosure, green propolis, wheatgrass, and mulberry leaves may be independently an unprocessed fresh material, or may be prepared by a process selected from the group consisting of a drying treatment, a grinding treatment, a chopping treatment, a comminuting treatment, and combinations thereof.

According to the present disclosure, before the extraction treatment, mulberry leaves may be subjected to a tea processing treatment using techniques well-known to those skilled in the art. Examples of the tea processing treatment may include, but are not limited to, withering (e.g., sun withering and hot air withering), stirring, panning, rolling, enzymatic oxidation, and drying.

In certain embodiments, the sun withering is conducted at a temperature ranging from 30° C. to 35° C., and the hot air withering is conducted at a temperature ranging from 35° C. to 38° C.

The present disclosure also provides a method for inhibiting the growth of cancer cells, which includes administering to a subject in need thereof the aforesaid anti-cancer composition.

As used herein, the term “subject” refers to any animal of interest, such as humans, monkeys, cows, sheep, horses, pigs, goats, dogs, cats, mice, and rats.

As used herein, the term “administration” or “administering” means introducing, providing or delivering a pre-determined active ingredient to a subject by any suitable routes to perform its intended function.

In certain embodiments, the cancer cells may be selected from the group consisting of brain cancer cells, kidney cancer cells, lung adenocarcinoma cells, skin cancer cells, breast cancer cells, liver cancer cells, bladder cancer cells, colorectal cancer cells, pancreatic cancer cells, blood cancer cells, and combinations thereof. In an exemplary embodiment, the cancer cells are brain cancer cells. In yet another exemplary embodiment, the cancer cells are kidney cancer cells.

According to the present disclosure, the anti-cancer composition may be employed in combination with photodynamic therapy (PDT) (abbreviated as green propolis-mediated PDT (GPDT)).

In certain embodiments, the photodynamic therapy may be conducted using a light irradiation that has a wavelength ranging from 400 nm to 700 nm. In an exemplary embodiment, the photodynamic therapy is conducted using a light irradiation that has a wavelength of 570 nm.

According to the present disclosure, the anti-cancer composition can effectively inhibit the growth of cancer cells and can exhibit an enhanced anti-cancer effect when further combined with PDT.

According to the present disclosure, the anti-cancer composition may be prepared in the form of a pharmaceutical composition. The pharmaceutical composition may be formulated into a dosage form suitable for oral administration, parenteral administration, or topical administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, scaffolds, and bandages.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intramuscular injection, intravenous injection, and subcutaneous injection.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

The dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials:

### 1. Sources of Green Propolis, Wheatgrass, and Mulberry Leaves

Green propolis was obtained from the beehives raised by Zhicheng Bee Farm (Taichung City, Wufeng District, Taiwan) and Mingyu Bee Farm (Taichung City, Waipu District, Taiwan). In addition, wheatgrass was obtained from the greenhouse of Fang-Gwann Biotechnology Co., Ltd. (Nantou County, Mingjian Township, Taiwan), and mulberry leaves were obtained from Quanming Ecological Education Sericulture Farm (Miaoli County, Shitan Township, Taiwan).

Before performing the following experiments, mulberry leaves were subjected to withering, stirring, panning, and rolling in sequence according to the tea pretreatment procedures well-known to those skilled in the art.

### 2. Source and Cultivation of Cell Lines

Human glioblastoma cell line U87 (BCRC 60360) and human renal cell carcinoma cell line 786-O (BCRC 60243) were purchased from the Bioresource Collection and Research Center (BCRC) of the Food Industry Research and Development Institute (FIRDI) (No. 331, Shih-Pin Rd., Hsinchu City 300, Taiwan).

U87 cells and 786-O cells were respectively grown in a 10-cm Petri dish containing Dulbecco's Modified Eagle's Medium (DMEM) (Gibco, Cat. No. 12100-038) supplemented with 10% fetal bovine serum (FBS) and 1% penicillin-streptomycin. The U87 cells and 786-O cells were cultivated in an incubator with culture conditions set at 37° C. and 5% CO2. Medium change was performed every two to three days. Cell passage was performed when the cultured cells reached 80% to 90% of confluence.

### Example 1. Preparation of Extract Mixture

An ethanol extract of green propolis was prepared according to the multi-level ultrasonic frequency conversion extraction procedures described in TW 1679015 B. Briefly, the green propolis described in section 1 of “General Experimental Materials” was mixed with 95% ethanol in a weight ratio of 1:3, and the resultant mixture was subjected to ultrasonication at a temperature ranging from 30° C. to 50° C. for 1 hour, followed by conducting filtration using a filter, so as to obtain a filtrate and a residue.

The residue was subjected to the abovementioned ethanol mixing-ultrasonication-filtration steps 3 times. Specifically, in the second ethanol mixing step, the residue was mixed with 95% ethanol in a weight ratio of 3:5. Next, all filtrates were collected, followed by conducting vacuum concentration to remove ethanol, so as to obtain the ethanol extract of green propolis.

The ethanol extract of green propolis was mixed with propylene glycol in a weight ratio of 1:1, and the resultant mixture was subjected to ultrasonication for 10 minutes, followed by conducting vacuum concentration at a temperature ranging from 55° C. to 65° C. to remove ethanol, so as to obtain a propylene glycol solution of green propolis extract.

Thereafter, the propylene glycol solution of green propolis extract was mixed with triglycerides in a weight ratio of 2:1, and the resultant mixture was subjected to ultrasonication for 10 minutes, followed by conducting a separation treatment using a thistle funnel. The lower layer thus formed was collected, so as to obtain a dewaxed propylene glycol solution containing green propolis extract (abbreviated as a GP extract).

In addition, the wheatgrass and the mulberry leaves described in section 1 of “General Experimental Materials” were respectively washed with water, followed by drying. Next, 100 g of a respective one of the dried wheatgrass and the dried mulberry leaves was mixed with 200 mL of 95% ethanol, and the resultant mixture was subjected to ultrasonication at a temperature ranging from 30° C. to 50° C. for 0.5 hour, followed by conducting filtration using a filter, so as to obtain a filtrate and a residue. The filtrates were collected to serve as an ethanol extract of wheatgrass and an ethanol extract of mulberry leaves, respectively.

The ethanol extract of wheatgrass and the ethanol extract of mulberry leaves were mixed in a weight ratio of 1:1, followed by being left standing at a temperature of 25° C. for 36 hours. The resultant mixture was subjected to filtration using a filter, so as to obtain a filtrate. The filtrate was mixed with propylene glycol in a weight ratio of 1:1, followed by conducting vacuum concentration to remove ethanol, so as to obtain a propylene glycol solution containing wheatgrass extract and mulberry leaves extract (abbreviated as a WM extract).

Thereafter, the GP extract and the WM extract were mixed in a weight ratio of 1:1, so as to obtain an extract mixture containing the GP extract and the WM extract (abbreviated as a GP-WM extract).

### Example 2. High Performance Liquid Chromatography (HPLC) Analysis

The GP-WM extract obtained in Example 1 was subjected to high performance liquid chromatography (HPLC) analysis which was entrusted to the Food Industry Research and Development Institute (FIRDI), Taiwan. The operating parameters and conditions for performing HPLC are summarized in Table 1 below.

TABLE 1

| | |
|---|---|
| HPLC instrument | Chromaster HPLC system (Hitachi) equipped with a |
| | pump (Hitachi, CM 5110) and a diode array detector |
| | (Hitachi, CM 5430) |
| Type of | C18 Column (Cosmosil ®) 5C18-MS-II) |
| chromatography | |
| column | |
| Size of chroma- | Length: 250 mm; |
| tography column | inner diameter: 4.6 mm |
| Detection | 320 nm |
| wavelength | |
| Mobile phase | Methanol (A)/0.4% phosphoric acid (B)(in methanol) |
| | (80:20, v/v) |
| Gradient elution | The mobile phase was conducted for 25 minutes as |
| | follows: A:B was 80:20 (v/v) during 0.1-9 minutes, |
| | A:B was 70:30 (v/v) during 9.1-12 minutes, A:B |
| | was 95:5 (v/v) during 12.1-15 minutes, A:B was |
| | 80:20 (v/v) during 15.1-25 minutes. |
| Flow rate of test | 0.3 mL/min |
| sample | |

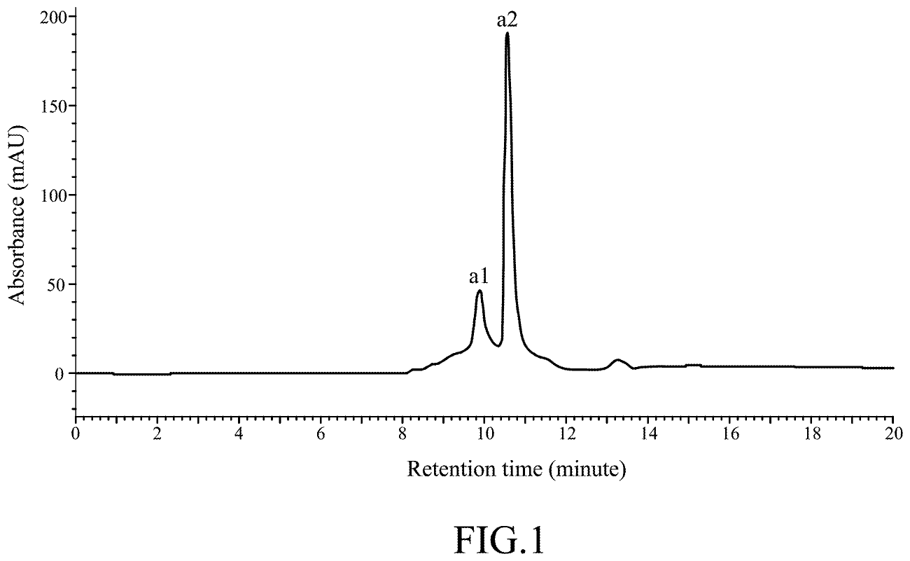

FIG. 1 shows the HPLC spectrum of the GP-WM extract. As shown in FIG. 1, there were two main peaks (i.e., peaks a1 and a2) during a 20-minute retention period, indicating that there were two major components in the GP-WM extract.

### Example 3. Evaluation for the Effect of GP-WM Extract in Inhibiting the Growth of Brain Cancer Cells

### A. Treatment of U87 Cells Using GP-WM Extract

The U87 cells prepared in section 2 of “General Experimental Materials” were incubated in a respective well of a 96-well culture plate containing 100 μL of Gibco Dulbecco's modified Eagle's medium (DMEM) (supplemented with 10% fetal bovine serum (FBS) and 1% penicillin-streptomycin) at 1×10⁴ cells/well, followed by cultivation in an incubator (37° C., 5% CO₂) for 24 hours.

Next, each of the cell cultures was treated with different amounts (i.e., 0.25 μL, 0.5 μL, 1 μL, 2 μL, 4 μL, and 8 μL) of a respective one of the GP extract, the WM extract, and the GP-WM extract prepared in Example 1, followed by cultivation in an incubator (37° C., 5% CO₂) for 24 hours.

Prior to the start of cultivation (i.e., on the 0th hour) and on the 24th hour after cultivation, each of the cell cultures was subjected to cell viability analysis using techniques well-known to those skilled in the art. Briefly, the liquid of XX in each well was removed, followed by adding a suitable amount of 3-[4,5-dimethylthiazol-2-yl]-2,5-diphenyltetrazoli-um bromide (MTT, 5 mg/mL). After cultivation in an incubator (37° C., 5% CO₂) for 24 hours, the liquid of XX in each well was removed, and the cells were washed twice with phosphate-buffered saline (PBS)(pH 7.4), followed by adding 50 μL of dimethyl sulfoxide (DMSO). The resultant reaction mixture was subsequently subjected to determination of absorbance at a wavelength of 570 nm (OD₅₇₀) using an ELISA reader (BioTek, Cat. No. Synergy HTX).

The cell viability percentage (%) was calculated using the following Equation (I):

A=(B/C)×100 (I)

where A=cell viability percentage (%)
B=OD₅₇₀ value determined on the 24^{th} hour after cultivation
C=OD₅₇₀ value determined on the 0^{th} hour (serving as a control group)

In addition, the median effective dose (ED50) was determined from the linear portion of the plotted dose-response curve by calculating the dose of active ingredient that exhibited a cell viability percentage of 50%.

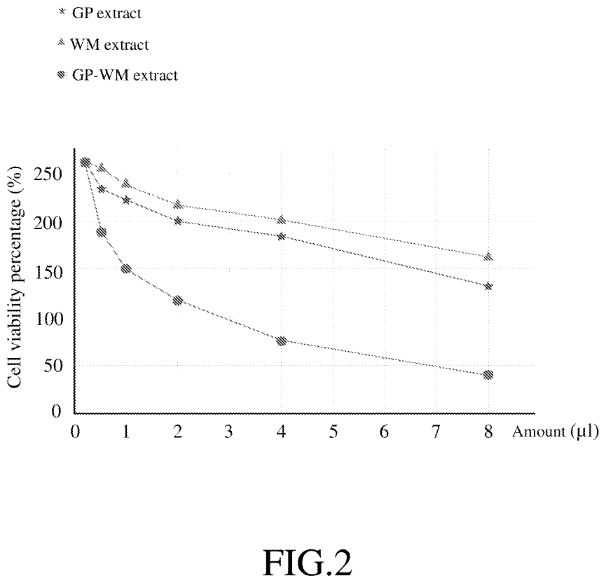

Referring to FIG. 2, at the same dose, the cell viability percentage determined for the GP-WM extract was significantly lower than those determined for the WM extract and the GP extract. Moreover, a significant dose-dependent effect of the GP-WM extract on the cell viability percentage was observed. In addition, the ED50 value of the GP-WM extract was 6.581 μL.

These results indicate that the GP-WM extract of the present disclosure is effective in inhibiting the growth of U87 cells.

B. Treatment of U87 Cells Using GP-WM Extract in Combination with Photodynamic Therapy (PDT)

Each of the cell cultures of the U87 cells was treated with different amounts (i.e., 0.25 μL, 0.5 μL, 1 μL, 2 μL, 4 μL, and 8 μL) of a respective one of the GP extract, the WM extract, and the GP-WM extract according to the method described in section A of this example, followed by cultivation in an incubator (37° C., 5% CO₂) for 24 hours. In addition, the cell cultures which received no treatment served as a control group.

Next, each of the cell cultures was subjected to PDT at a wavelength of 570 nm using a fluorescent lamp for 24 hours. Subsequently, each of the cell cultures was subjected to cell viability analysis and determination of ED50 value according to the methods described in section A of this example.

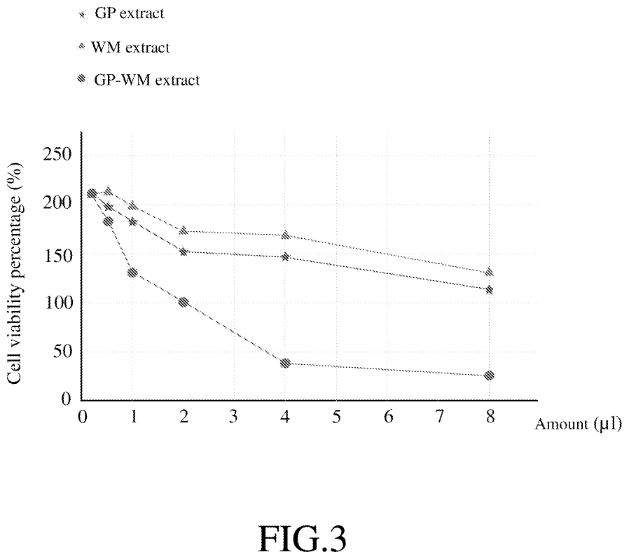

Referring to FIG. 3, at the same dose, the cell viability percentage determined for the GP-WM extract was significantly lower than those determined for the WM extract and the GP extract. Moreover, a significant dose-dependent effect of the GP-WM extract on the cell viability percentage was observed. In addition, the ED50 value of the GP-WM extract was 3.5 μL.

These results indicate that the GP-WM extract of the present disclosure can exhibit satisfactory efficacy in inhibiting the growth of U87 cells, and this efficacy can be enhanced when the GP-WM extract is used in combination with PDT.

### Example 4. Evaluation for the Effect of GP-WM Extract in Inhibiting the Growth of Kidney Cancer Cells

The 786-O cells prepared in section 2 of “General Experimental Materials” were incubated in a respective well of a 96-well culture plate containing 100 μL of DMEM (supplemented with 10% FBS and 1% penicillin-streptomycin) at 1×10⁴ cells/well, followed by cultivation in an incubator (37° C., 5% CO₂) for 24 hours.

Next, each of the cell cultures was treated with different amounts (i.e., 0.125 μL, 0.25 μL, 0.5 μL, 1 μL, 2 μL, 4 μL, and 8 μL) of the GP-WM extract prepared in Example 1, followed by cultivation in an incubator (37° C., 5% CO₂) for 24 hours. In addition, the cell cultures which received no treatment served as a control group.

Subsequently, each of the cell cultures was subjected to cell viability analysis and determination of ED50 value according to the methods described in section A of Example 3.

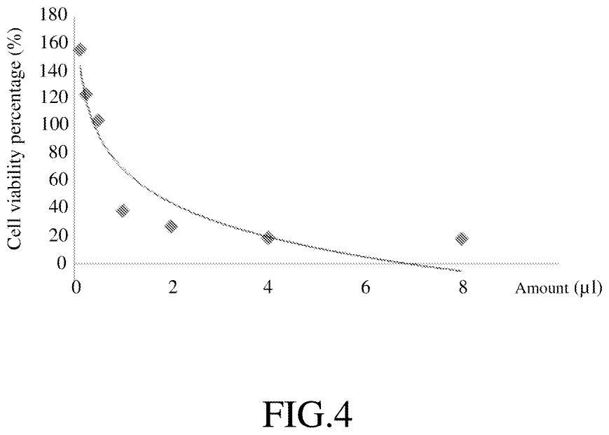

Referring to FIG. 4, the GP-WM extract of the present disclosure exhibited a significant dose-dependent cytotoxic effect on 786-O cells, and the ED50 value of the GP-WM extract was 1.728 μL. These results indicate that the GP-WM extract of the present disclosure is effective in inhibiting the growth of 786-O cells.

Summarizing the above test results, it is clear that the extract mixture (i.e., the GP-WM extract) containing the ethanol extract of green propolis, the ethanol extract of wheatgrass, and the ethanol extract of mulberry leaves is capable of inhibiting the growth of cancer cells (such as brain cancer cells and kidney cancer cells), and when used in combination with PDT, can synergistically exhibit an improved efficacy.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An anti-cancer composition, comprising an ethanol extract of green propolis, an ethanol extract of wheatgrass, and an ethanol extract of mulberry leaves.

2. The anti-cancer composition as claimed in claim 1, wherein each of the ethanol extract of green propolis, the ethanol extract of wheatgrass, and the ethanol extract of mulberry leaves is dissolved in propylene glycol.

3. The anti-cancer composition as claimed in claim 1, wherein a weight ratio of the ethanol extract of green propolis, the ethanol extract of wheatgrass, and the ethanol extract of mulberry leaves ranges from 1:0.1:0.1 to 1:1:1.

4. A method for inhibiting the growth of cancer cells, comprising administering to a subject in need thereof an anti-cancer composition as claimed in claim 1.

5. The method as claimed in claim 4, wherein the cancer cells are selected from the group consisting of brain cancer cells, kidney cancer cells, lung adenocarcinoma cells, skin cancer cells, breast cancer cells, liver cancer cells, bladder cancer cells, colorectal cancer cells, pancreatic cancer cells, blood cancer cells, and combinations thereof.

6. The method as claimed in claim 4, wherein the anti-cancer composition is employed in combination with photodynamic therapy.

7. The method as claimed in claim 6, wherein the photodynamic therapy is conducted using a light irradiation having a wavelength ranging from 400 nm to 700 nm.

8. The method as claimed in claim 4, wherein the anti-cancer composition is formulated as a pharmaceutical composition.

9. The method as claimed in claim 8, wherein the pharmaceutical composition is in a dosage form selected from the group consisting of an oral dosage form, a parenteral dosage form, and a topical dosage form.
